(19) United States
(12) Patent Application Publication
(10) Pub. No.: US 2024/0123462 A1
(43) Pub. Date: Apr. 18, 2024

(54) ATOMIZATION MODULE

(71) Applicant: HCMed Innovations Co., LTD., Taipei City (TW)

(72) Inventors: CHIA-CHIEN CHANG, New Taipei City (TW); HSIN-YI PAI, New Taipei City (TW); CHUN-CHIA JUAN, New Taipei City (TW)

(21) Appl. No.: 18/486,169

(22) Filed: Oct. 13, 2023

Related U.S. Application Data
(60) Provisional application No. 63/416,593, filed on Oct. 16, 2022.

(30) Foreign Application Priority Data
Sep. 13, 2023 (CN) 202311175135.7

(51) Int. Cl. B05B 17/00; A61M 11/00

(52) U.S. Cl. CPC B05B 17/0646; A61M 11/00

(57) ABSTRACT

An atomization module includes a base, an auxiliary fixing member, an atomization member, a piezoelectric element, a conductive member, and a waterproof member. The base includes a support portion and an outer annular portion. The support portion has a first opening, the outer annular portion surrounds the support portion, and the support portion protrudes relative to the outer annular portion. The auxiliary fixing member is disposed on the support portion, and has a second opening. The second opening is opposite to the first opening. The atomization member is disposed between the support portion and the auxiliary fixing member. The piezoelectric element is disposed on the outer annular portion, and the piezoelectric element and the atomization member are disposed on a same side of the base. The conductive member is electrically connected to the piezoelectric element. The waterproof member at least partially covers the piezoelectric element.

### CROSS-REFERENCE TO RELATED PATENT APPLICATION

This application claims the benefit of priorities to the U.S. Provisional Patent Application Ser. No. 63/416,593, filed on Oct. 16, 2022, and China Patent Application No. 202311175135.7, filed on Sep. 13, 2023, in the People's Republic of China. The entire content of each of the above identified applications is incorporated herein by reference.

Some references, which may include patents, patent applications and various publications, may be cited and discussed in the description of this disclosure. The citation and/or discussion of such references is provided merely to clarify the description of the present disclosure and is not an admission that any such reference is “prior art” to the disclosure described herein. All references cited and discussed in this specification are incorporated herein by reference in their entireties and to the same extent as if each reference was individually incorporated by reference.

### FIELD OF THE DISCLOSURE

The present disclosure relates to an atomization module, and more particularly to an atomization module that has a waterproof structure.

### BACKGROUND OF THE DISCLOSURE

Through an internal atomization assembly, an atomizer can atomize a liquid medicine into vapor for spraying. After a piezoelectric element is electrically conducted, an atomization member inside the atomization assembly can generate vibration, so as to convert the liquid medicine into vapor containing droplets. During use of the atomizer, residues of the liquid medicine may remain in the atomizer. In addition, the atomizer needs to be washed with water or sanitized at a high temperature. In the above-mentioned processes, water vapor is likely to seep into the atomizer and cause short-circuiting of internal electronic components (e.g., the piezoelectric element).

Therefore, how to enhance the waterproof performance of the atomizer through improvements in structural design, so as to overcome the above-mentioned problems, has become one of the important issues to be solved in the related art.

### SUMMARY OF THE DISCLOSURE

In response to the above-referenced technical inadequacies, the present disclosure provides an atomization module, so as to solve the problem of water vapor easily seeping into a conventional atomizer and causing short-circuiting of internal electronic components.

In order to solve the above-mentioned problems, one of the technical aspects adopted by the present disclosure is to provide an atomization module, which includes a base, an auxiliary fixing member, an atomization member, a piezoelectric element, a conductive member, and a waterproof member. The base includes a support portion and an outer annular portion. The support portion has a first opening, the outer annular portion surrounds the support portion, and the support portion protrudes relative to the outer annular portion. The auxiliary fixing member is disposed on the support portion. The auxiliary fixing member has a second opening, and the second opening is opposite to the first opening. The atomization member is disposed between the support portion and the auxiliary fixing member. The piezoelectric element is disposed on the outer annular portion, and the piezoelectric element and the atomization member are disposed on a same side of the base. The conductive member is electrically connected to the piezoelectric element. The waterproof member at least partially covers the piezoelectric element.

In order to solve the above-mentioned problems, another one of the technical aspects adopted by the present disclosure is to provide an atomization module, which includes an atomization structure, and a first waterproof member and a second waterproof member that are separable from one another. The atomization structure includes a base, an auxiliary fixing member, an atomization member, a piezoelectric element, and a conductive member. The base includes a support portion and an outer annular portion. The support portion has a first opening, the outer annular portion surrounds the support portion, and the support portion protrudes relative to the outer annular portion. The auxiliary fixing member is disposed on the support portion. The auxiliary fixing member has a second opening, and the second opening is opposite to the first opening. The atomization member is disposed between the support portion and the auxiliary fixing member. The piezoelectric element is disposed on the outer annular portion, and the piezoelectric element and the atomization member are disposed on a same side of the base. The conductive member is electrically connected to the piezoelectric element. The atomization structure is covered top and bottom by the first waterproof member and the second waterproof member.

Therefore, in the atomization module provided by the present disclosure, by virtue of “the waterproof member at least partially covering the piezoelectric element” and “the first waterproof member and the second waterproof member being separable from one another, and the atomization structure being covered top and bottom by the first waterproof member and the second waterproof member,” the waterproof performance of the atomization module can be enhanced.

These and other aspects of the present disclosure will become apparent from the following description of the embodiment taken in conjunction with the following drawings and their captions, although variations and modifications therein may be affected without departing from the spirit and scope of the novel concepts of the disclosure.

### BRIEF DESCRIPTION OF THE DRAWINGS

The described embodiments may be better understood by reference to the following description and the accompanying drawings, in which:

FIG. 1 is a schematic view of an atomization structure according to the present disclosure;

FIG. 2 is a schematic exploded view of the atomization structure according to the present disclosure;

FIG. 3 is a schematic view of an atomization module according to a first embodiment of the present disclosure;

FIG. 4 is a schematic cross-sectional view taken along line IV-IV of FIG. 3;

FIG. 5 is a schematic view of an atomization module according to a second embodiment of the present disclosure;

FIG. 6 is a schematic cross-sectional view taken along line VI-VI of FIG. 5;

FIG. 7 is a schematic view of an atomization module according to a third and a fourth embodiment of the present disclosure;

FIG. 8 is another schematic view of the atomization module according to the third and the fourth embodiment of the present disclosure;

FIG. 9 is a schematic cross-sectional view of the atomization module according to the third embodiment of the present disclosure;

FIG. 10 is a schematic exploded view of an atomization structure and a spacer according to the fourth embodiment of the present disclosure;

FIG. 11 is another schematic exploded view of the atomization structure and the spacer according to the fourth embodiment of the present disclosure;

FIG. 12 is a schematic cross-sectional view of the atomization module according to the fourth embodiment of the present disclosure;

FIG. 13 is a schematic view of an atomization module according to a fifth embodiment of the present disclosure;

FIG. 14 is a schematic cross-sectional view taken along line XIV-XIV of FIG. 13;

FIG. 15 is a schematic view of an atomization module according to a sixth embodiment of the present disclosure;

FIG. 16 is a schematic exploded view of the atomization module according to the sixth embodiment of the present disclosure;

FIG. 17 is a schematic view of an atomization module according to a seventh embodiment of the present disclosure;

FIG. 18 is another schematic view of the atomization module according to the seventh embodiment of the present disclosure; and

FIG. 19 is a schematic exploded view of the atomization module according to the seventh embodiment of the present disclosure.

### DETAILED DESCRIPTION OF THE EXEMPLARY EMBODIMENTS

The present disclosure is more particularly described in the following examples that are intended as illustrative only since numerous modifications and variations therein will be apparent to those skilled in the art. Like numbers in the drawings indicate like components throughout the views. As used in the description herein and throughout the claims that follow, unless the context clearly dictates otherwise, the meaning of “a,” “an” and “the” includes plural reference, and the meaning of “in” includes “in” and “on.” Titles or subtitles can be used herein for the convenience of a reader, which shall have no influence on the scope of the present disclosure.

The terms used herein generally have their ordinary meanings in the art. In the case of conflict, the present document, including any definitions given herein, will prevail. The same thing can be expressed in more than one way. Alternative language and synonyms can be used for any term(s) discussed herein, and no special significance is to be placed upon whether a term is elaborated or discussed herein. A recital of one or more synonyms does not exclude the use of other synonyms. The use of examples anywhere in this specification including examples of any terms is illustrative only, and in no way limits the scope and meaning of the present disclosure or of any exemplified term. Likewise, the present disclosure is not limited to various embodiments given herein. Numbering terms such as “first,” “second” or “third” can be used to describe various components, signals or the like, which are for distinguishing one component/signal from another one only, and are not intended to, nor should be construed to impose any substantive limitations on the components, signals or the like.

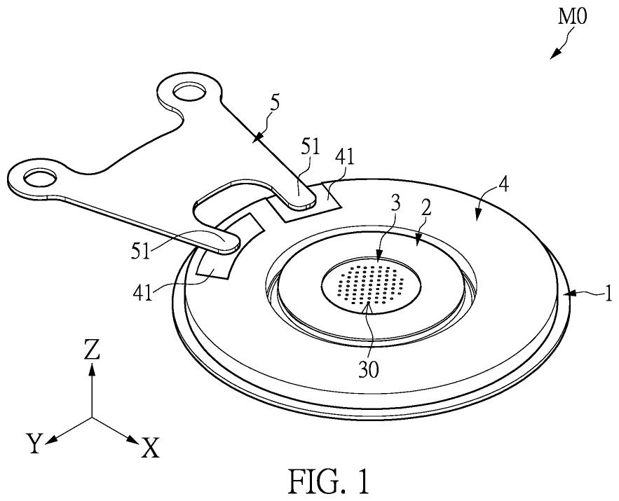

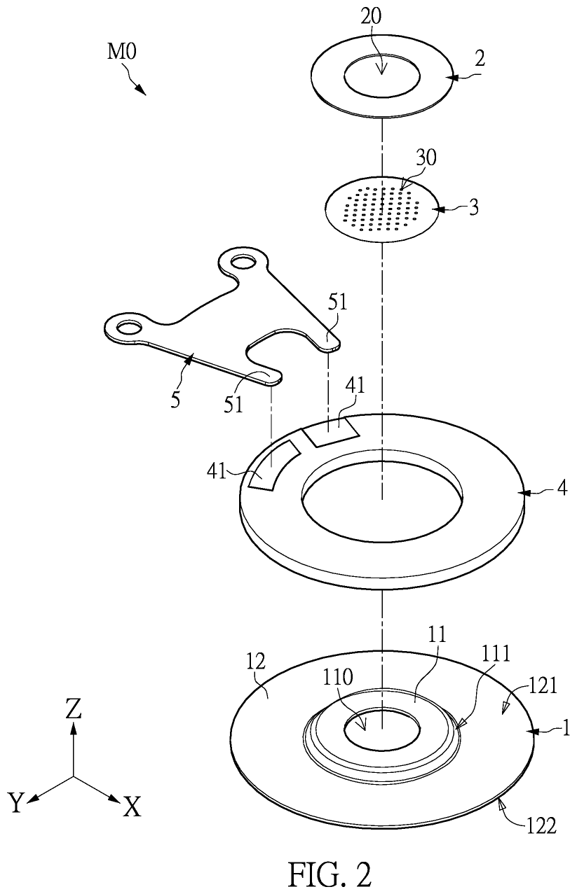

Referring to FIG. 1 and FIG. 2, FIG. 1 is a schematic view of an atomization structure according to the present disclosure, and FIG. 2 is a schematic exploded view of the atomization structure according to the present disclosure. The present disclosure provides an atomization structure M0, which includes a base 1, an auxiliary fixing member 2, an atomization member 3, a piezoelectric element 4, and a conductive member 5.

The base 1 includes a support portion 11 and an outer annular portion 12. The support portion 11 has a first opening 110, the outer annular portion 12 surrounds the support portion 11, and the support portion 11 protrudes relative to the outer annular portion 12. The auxiliary fixing member 2 is disposed on the support portion 11. The auxiliary fixing member 2 has a second opening 20, and the second opening 20 is opposite to the first opening 110. The atomization member 3 is disposed between the support portion 11 and the auxiliary fixing member 2. The atomization member 3 has a plurality of through holes 30, and the through holes 30, the first opening 110, and the second opening 20 are in spatial communication with each other.

The piezoelectric element 4 can be an annular structure, and is disposed on the outer annular portion 12. The piezoelectric element 4 and the atomization member 3 are disposed on the same side of the base 1. Specifically, the outer annular portion 12 has a first surface 121 and a second surface 122 that are opposite to each other, and the piezoelectric element 4 and the auxiliary fixing member 2 are disposed on the first surface 121. For example, the conductive member 5 can be a flexible printed circuit (FPC) or a flexible flat cable, but is not limited thereto. Two electrode portions 41 are disposed on a top surface of the piezoelectric element 4, and the conductive member 5 has two connection portions 51. Through soldering the two connection portions 51 respectively to the two electrode portions 41, the conductive member 5 can be electrically connected to the piezoelectric element 4. However, a manner by which the connection portions 51 are connected to the electrode portions 41 is not limited in the present disclosure. Through the conductive member 5, the piezoelectric element 4 can input a voltage and drive the atomization member 3 to vibrate. Accordingly, when passing through the through holes 30 of the vibrating atomization member 3, a liquid medicine in an atomizer (not shown) can be sprayed to an external environment in the form of vapor.

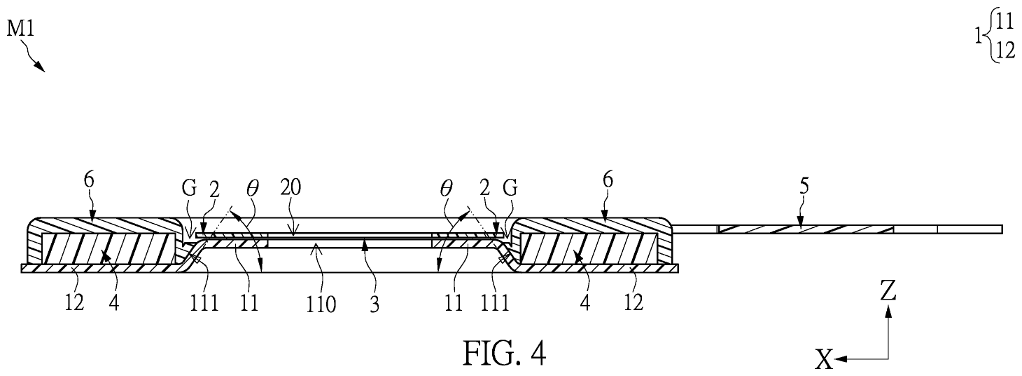

In the present disclosure, the support portion 11 is configured to be protruding, such that the atomization member 3 disposed above the support portion 11 can have an enhanced vibration effect when the piezoelectric element 4 is powered. For example, a height difference of between 200 μm and 1,000 μm is defined between the support portion 11 and the outer annular portion 12, or an inclined support surface 111 is disposed between the support portion 11 and the outer annular portion 12. The inclined support surface 111 has an inclination angle θ relative to a horizontal plane (as shown in FIG. 4), and the inclination angle θ ranges between 20° C. and 80° C. By configuring the support portion 11 to be protruding in the present disclosure, an amplitude transmitted to the atomization member 3 after powering of the piezoelectric element 4 can be enlarged, and the atomization member 3 disposed above the support portion 11 can have an optimized vibration efficiency when the piezoelectric element 4 is powered.

It should be noted that an atomization module in each embodiment of the present disclosure includes the atomization structure M0 shown in FIG. 1 and FIG. 2, and the detailed structure thereof will not be reiterated herein. In addition, the atomization module further includes a waterproof member that at least partially covers the piezoelectric element 4. In different embodiments, the waterproof member may have different configurations, which will be illustrated below.

### First Embodiment

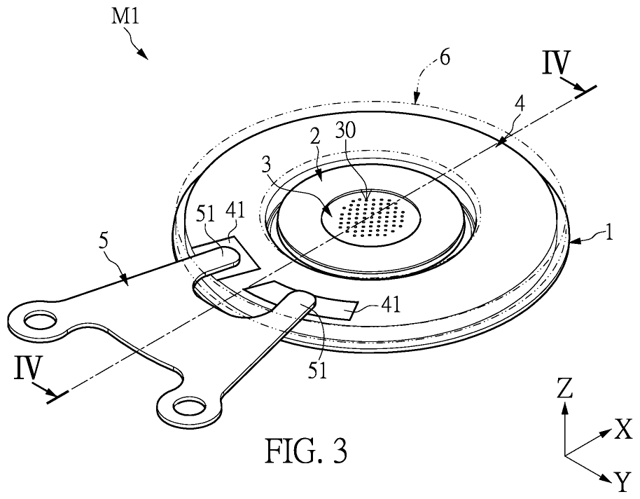

Referring to FIG. 3 and FIG. 4, FIG. 3 is a schematic view of an atomization module according to a first embodiment of the present disclosure, and FIG. 4 is a schematic cross-sectional view taken along line IV-IV of FIG. 3. The first embodiment of the present disclosure provides an atomization module M1, which includes the base 1, the auxiliary fixing member 2, the atomization member 3, the piezoelectric element 4, the conductive member 5, and a waterproof member 6. The base 1, the auxiliary fixing member 2, the atomization member 3, the piezoelectric element 4, and the conductive member 5 form the atomization structure M0 as shown in FIG. 1 and FIG. 2.

The waterproof member 6 is disposed on the outer annular portion 12 of the base 1, and completely covers the piezoelectric element 4. Since the two connection portions 51 of the conductive member 5 are electrically connected to the two electrode portions 41 of the piezoelectric element 4, the two electrode portions 41 are also covered by the waterproof member 6 when the waterproof member 6 completely covers the piezoelectric element 4. Specifically, the waterproof member 6 is attached to the inclined support surface 111. In other words, the waterproof member 6 will extend to and be filled in a gap G between the inclined support surface 111 of the support portion 11 and the piezoelectric element 4.

For example, the waterproof member 6 is a waterproof soft gel. The waterproof member 6 can be formed by plating or coating, or by thermoforming or injection molding, and completely covers the piezoelectric element 4. There is no limitation as to the material of the waterproof member 6 and how the waterproof member 6 is formed in the present disclosure.

In the first embodiment, since the waterproof member 6 covers the piezoelectric element 4 and the two connection portions 51 of the conductive member 5, and further extends to the gap G between the inclined support surface 111 and the piezoelectric element 4, seepage of water vapor can be prevented, and short-circuiting caused by electrical conduction of a positive electrode (the top surface) and a negative electrode (a bottom surface) of the piezoelectric element 4 does not occur, thereby achieving waterproof and insulating effects. Furthermore, since the waterproof member 6 only needs to be disposed on one side of the base 1, and covers the piezoelectric element 4 and soldered portions of the electrode portions 41 and the connection portions 51, a manufacturing process can be simplified, and the difficulty in an overmolding process of the waterproof member 6 can be reduced. More specifically, since the waterproof member 6 of the atomization module M1 in the first embodiment is formed by plating or coating, a bonding strength between the waterproof member 6 and the atomization structure M0 is high. When the atomization module M1 is assembled inside the atomizer, the atomization module M1 can be fixed without needing to apply a considerable clamping force to two silica gel gaskets (not shown) respectively disposed on top and bottom positions of the atomization module M1.

### Second Embodiment

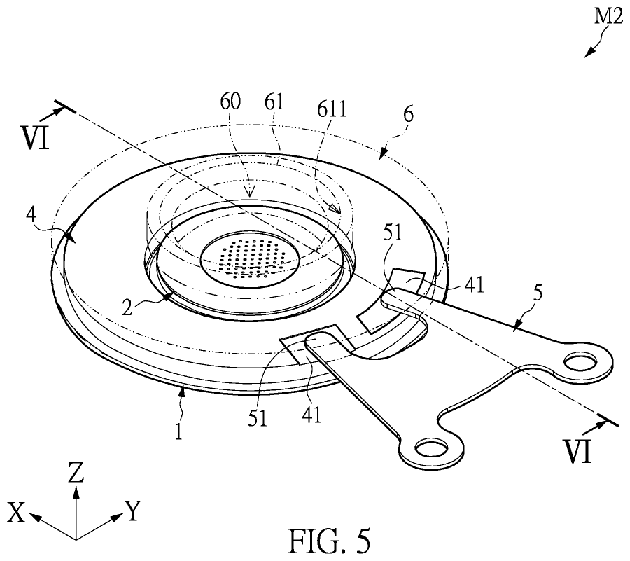

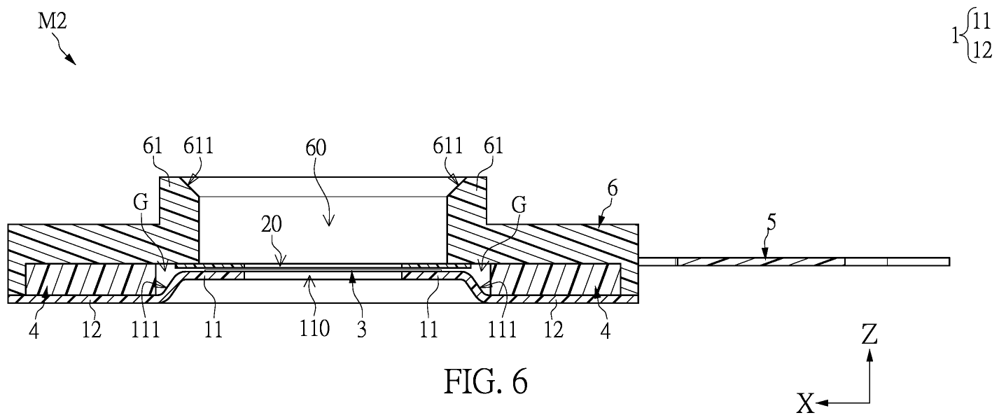

Referring to FIG. 5 and FIG. 6, FIG. 5 is a schematic view of an atomization module according to a second embodiment of the present disclosure, and FIG. 6 is a schematic cross-sectional view taken along line VI-VI of FIG. 5. The second embodiment of the present disclosure provides an atomization module M2, which includes the base 1, the auxiliary fixing member 2, the atomization member 3, the piezoelectric element 4, the conductive member 5, and the waterproof member 6. The base 1, the auxiliary fixing member 2, the atomization member 3, the piezoelectric element 4, and the conductive member 5 form the atomization structure M0 as shown in FIG. 1 and FIG. 2.

The waterproof member 6 is disposed on the outer annular portion 12 of the base 1, and completely covers the piezoelectric element 4 and the two connection portions 51 of the conductive member 5. In addition, a portion of the auxiliary fixing member 2 is also covered by the waterproof member 6 (as shown in FIG. 6). It should be noted that the gap G at this time can be filled or not filled according to practical requirements.

The waterproof member 6 has a third opening 60, and the third opening 60 is in spatial communication with the second opening 20 of the auxiliary fixing member 2. An annular convex wall 61 of the waterproof member 6 is formed along a periphery of the third opening 60, and an inner section surface 611 is formed at an inner periphery of the annular convex wall 61. When the atomization module M2 is disposed inside the atomizer, the annular convex wall 61 protrudes and extends downwardly, and is mated to a matching structure inside the atomizer. For example, the annular convex wall 61 can be engaged with an opening inside the atomizer (not shown).

For example, through injection molding or extrusion, the waterproof member 6 can cover the piezoelectric element 4. Similarly, the waterproof member 6 only needs to be disposed on one side of the base 1, and covers the piezoelectric element 4 and the soldered portions of the electrode portions 41 and the connection portions 51. In this way, the manufacturing process can be simplified, and the difficulty in the overmolding process of the waterproof member 6 can be reduced.

In another embodiment, the atomization module M2 can further include an adhesive (not shown). Through a dispensing process, the adhesive can be filled between the base 1, the auxiliary fixing member 2, the piezoelectric element 4, and the waterproof member 6. Specifically, the adhesive is not only filled in the gap G between the piezoelectric element 4 and the inclined support surface 111 of the base 1, but is also distributed between the piezoelectric element 4 and the waterproof member 6 and between the waterproof member 6 and the auxiliary fixing member 2, so as to improve a structural strength of the atomization module M2.

After use, the atomizer will be disassembled and washed, and the atomization module inside the atomizer also needs to be sanitized in boiling hot water. During the boiling and sanitization process, a gap between the waterproof member 6 and the atomization structure M0 will be increased due to thermal expansion of the air. As a result, an adhesive strength between the waterproof member 6 and the atomization structure M0 is reduced, such that a bonding surface between the waterproof member 6 and the atomization structure M0 peels off Therefore, the gap between the waterproof member 6 and the atomization structure M0 is configured to be filled with the adhesive, such that the gap is capable of blocking the water vapor, and is prevented from being increased by the thermally-expanded air during the boiling and sanitization process (which may damage the structure of the atomization module M2).

### Third Embodiment

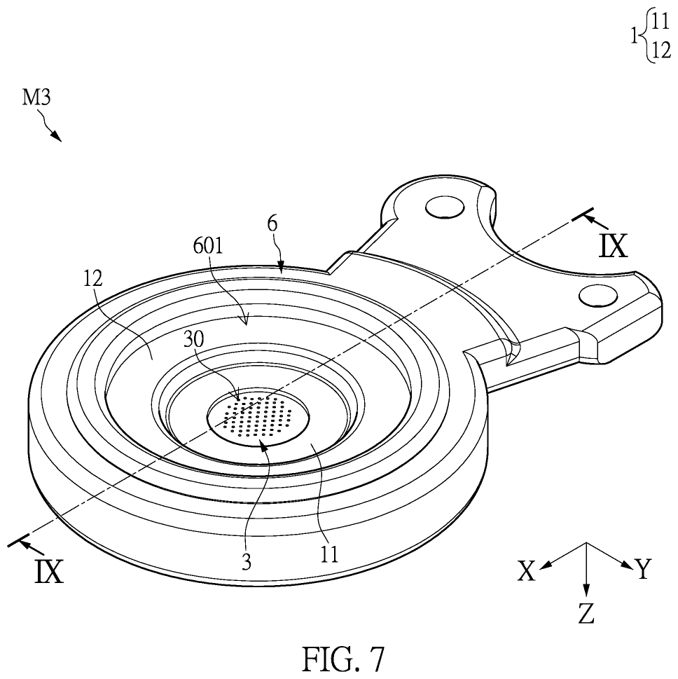

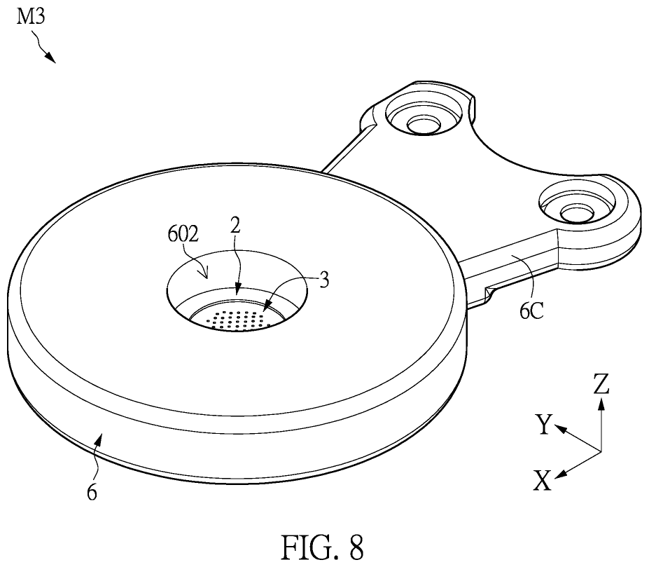

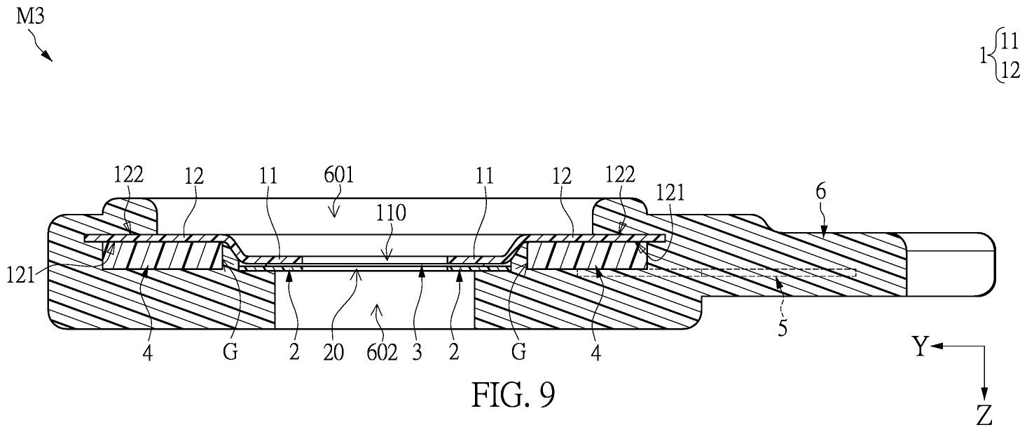

Referring to FIG. 7 to FIG. 9, FIG. 7 and FIG. 8 are schematic views of an atomization module according to a third embodiment of the present disclosure from different viewing angles, and FIG. 9 is a schematic cross-sectional view of the atomization module according to the third embodiment of the present disclosure. The third embodiment of the present disclosure provides an atomization module M3, which includes the base 1, the auxiliary fixing member 2, the atomization member 3, the piezoelectric element 4, the conductive member 5, and the waterproof member 6. The base 1, the auxiliary fixing member 2, the atomization member 3, the piezoelectric element 4, and the conductive member 5 form the atomization structure M0 as shown in FIG. 1 and FIG. 2.

The waterproof member 6 has a third opening 601 and a fourth opening 602. The third opening 601 is in spatial communication with the second opening 20 of the auxiliary fixing member 2, and the fourth opening 602 is in spatial communication with the first opening 110 of the base 1, such that the atomization member 3 is exposed from the waterproof member 6. For example, the waterproof member 6 can be formed by injection molding, but is not limited thereto.

The waterproof member 6 completely covers the piezoelectric element 4 and the conductive member 5 (a portion 6C of the waterproof member 6 extends to cover the conductive member 5), and partially covers the base 1 and the auxiliary fixing member 2. As shown in FIG. 9, the waterproof member 6 is filled in the gap G between the base 1, the auxiliary fixing member 2, and the piezoelectric element 4. In this way, the waterproof member 6 can prevent the liquid medicine from overflowing to the piezoelectric element 4 in a liquid form or a vapor form during vibration of the atomization member 3, and short-circuiting caused by electrical conduction of the positive electrode (the top surface) and the negative electrode (the bottom surface) of the piezoelectric element 4 does not occur, thereby achieving an electrical insulating effect.

The outer annular portion 12 has the first surface 121 and the second surface 122 that are opposite to each other, and the piezoelectric element 4 and the auxiliary fixing member 2 are disposed on the first surface 121. When the waterproof member 6 covers the base 1, the waterproof member 6 extends from the first surface 121 to the second surface 122 of the outer annular portion 12, and partially covers the second surface 122. Specifically, the waterproof member 6 at least covers one half of the second surface 122, and at least covers one third of a surface of the auxiliary fixing member 2. Accordingly, the stability of the waterproof member 6 when covering the atomization structure M0 can be increased, thereby improving a structural strength of the atomization module M3 and prolonging a service life of the atomization module M3.

### Fourth Embodiment

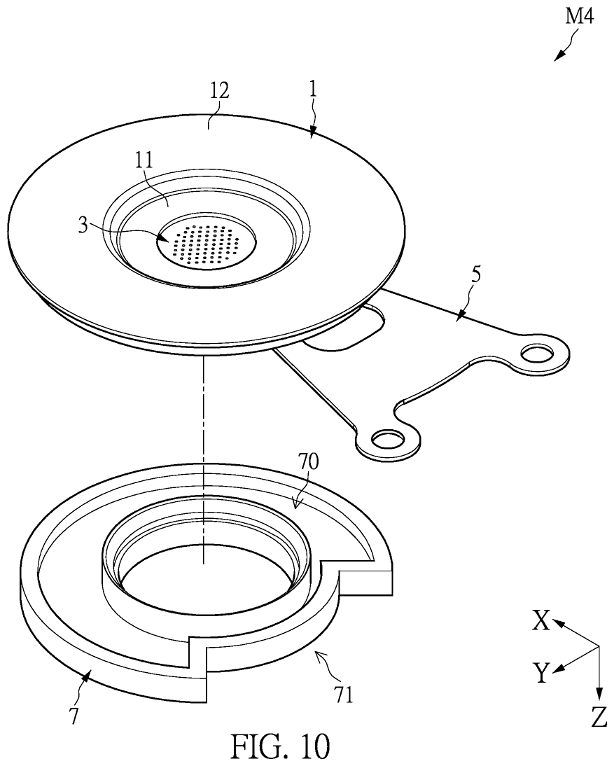

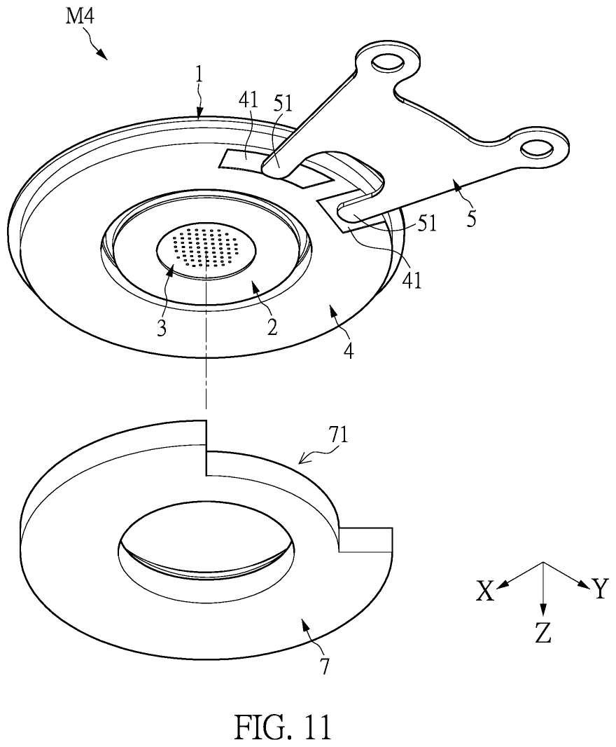

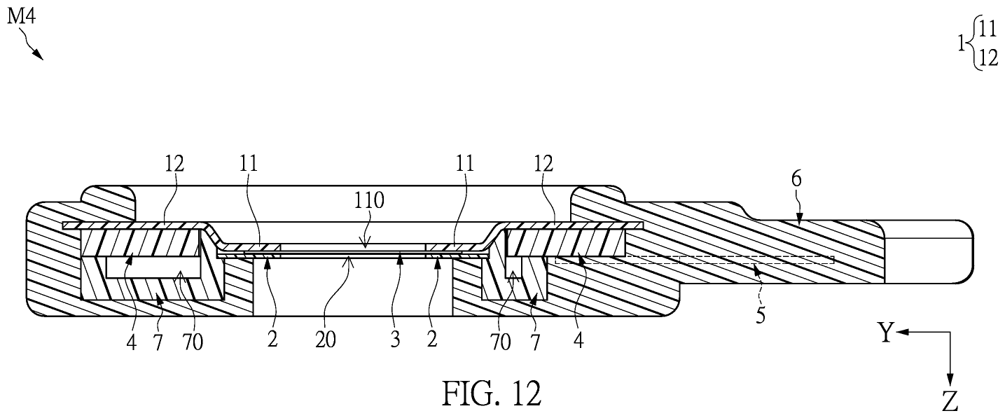

Referring to FIG. 10 to FIG. 12, FIG. 10 is a schematic exploded view of an atomization structure and a spacer according to a fourth embodiment of the present disclosure, FIG. 11 is another schematic exploded view of the atomization structure and the spacer according to the fourth embodiment of the present disclosure, and FIG. 12 is a schematic cross-sectional view of the atomization module according to the fourth embodiment of the present disclosure. Since an atomization module M4 of the fourth embodiment in the present disclosure and the atomization module M3 of the third embodiment have a same outer appearance and are similar in terms of internal composition structure (as shown in FIG. 7 and FIG. 8), similarities therebetween will not be reiterated herein.

The main difference between the third embodiment and the fourth embodiment is that the atomization module M4 of the fourth embodiment further includes a spacer 7. During formation of the waterproof member 6, the spacer 7 is synchronously inserted in the waterproof member 6 by injection molding. However, there is no limitation as to how the spacer 7 is formed in the present disclosure. The waterproof member 6 completely covers the piezoelectric element 4, the conductive member 5, and the spacer 7. The spacer 7 can be a gel that has a waterproof property, and is an annular structure. The composition material of the spacer 7 can be the same as or different from that of the waterproof member 6. The composition material of the spacer 7 is not limited in the present disclosure. The spacer 7 and the piezoelectric element 4 are disposed on the same side of the base 1. The spacer 7 covers the piezoelectric element 4, and partially covers the auxiliary fixing member 2. A groove 70 is formed on a side of the spacer 7 that faces toward the base 1. When the spacer 7 covers the piezoelectric element 4, an interior of the groove 70 is defined as an enclosed space, and only a portion of the top surface of the piezoelectric element 4 (i.e., a surface having the electrode portions 41) is in contact with the spacer 7.

Furthermore, the spacer 7 has an opening portion 71. When the spacer covers the piezoelectric element 4, the opening portion 71 is correspondingly disposed on the soldered portions of the electrode portions 41 of the piezoelectric element 4 and the connection portions 51 of the conductive member 5. Therefore, the electrode portions 41 and the connection portions 51 are not covered by the spacer 7. Through the structural design of the groove 70, the piezoelectric element 4 can have a sufficient space for vibration, and is thus not restricted by the waterproof member 6 when generating vibration.

### Fifth Embodiment

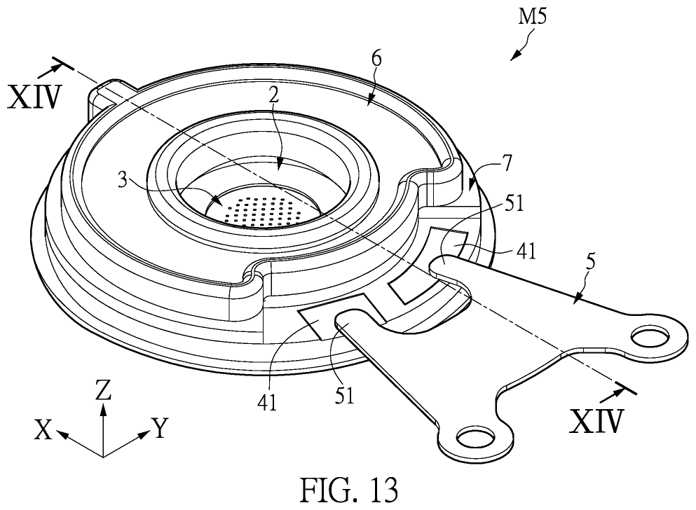

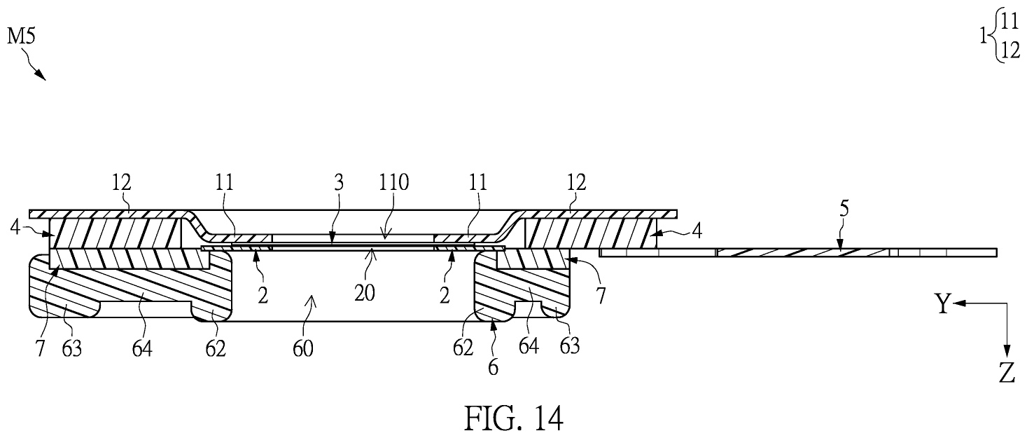

Referring to FIG. 13 and FIG. 14, FIG. 13 is a schematic view of an atomization module according to a fifth embodiment of the present disclosure, and FIG. 14 is a schematic cross-sectional view taken along line XIV-XIV of FIG. 13. The fifth embodiment of the present disclosure provides an atomization module M5, which includes the atomization structure M0, the waterproof member 6, and the spacer 7. The atomization structure M0 includes the base 1, the auxiliary fixing member 2, the atomization member 3, the piezoelectric element 4, and the conductive member 5, and the specific structure thereof is as shown in FIG. 1 and FIG. 2.

The structure and the configuration of the spacer 7 of the fifth embodiment are similar to those of the spacer 7 of the fourth embodiment. The waterproof member 6 covers the spacer 7 from above, and the spacer 7 is disposed between the waterproof member 6 and the piezoelectric element 4. The spacer 7 covers the piezoelectric element 4. Compared with the fourth embodiment, the waterproof member 6 of the fifth embodiment only partially covers the piezoelectric element 4.

The spacer 7 has the opening portion 71, which is correspondingly disposed on the soldered portions of the electrode portions 41 of the piezoelectric element 4 and the connection portions 51 of the conductive member 5. When the spacer 7 covers the piezoelectric element 4, most of the spacer 7 covers the top surface of the piezoelectric element 4 (i.e., the surface having the electrode portions 41), but does not cover the electrode portions 41 of the piezoelectric element 4 and the connection portions 51 of the conductive member 5.

In the fifth embodiment, the waterproof member 6 corresponds in shape to the spacer 7, and the waterproof member 6 also has a notch that corresponds to the opening portion 71. When the waterproof member 6 is disposed above the spacer 7, the electrode portions 41 of the piezoelectric element 4 and the connection portions 51 of the conductive member 5 are not covered by the waterproof member 6.

As shown in FIG. 14, the waterproof member 6 includes a first waterproof wall 62, a second waterproof wall 63, and a mid-portion 64. The first waterproof wall 62 is configured to surround the third opening 60, the second waterproof wall 63 surrounds an outer periphery of the first waterproof wall 62, and the mid-portion 64 is connected between the first waterproof wall 62 and the second waterproof wall 63. A thickness of the mid-portion 64 is less than a thickness of the first waterproof wall 62 and a thickness of the second waterproof wall 63. In the present embodiment, the spacer 7 can be completely or partially filled between the first waterproof wall 62 and the second waterproof wall 63. Specifically, the first waterproof wall 62 extends toward the atomization member 3 along an inner side of the spacer 7, and partially covers the auxiliary fixing member 2.

### Sixth Embodiment

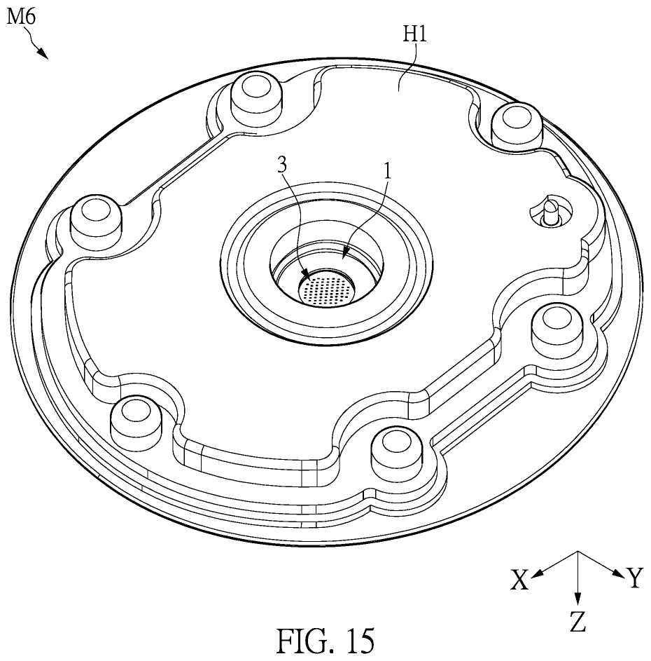

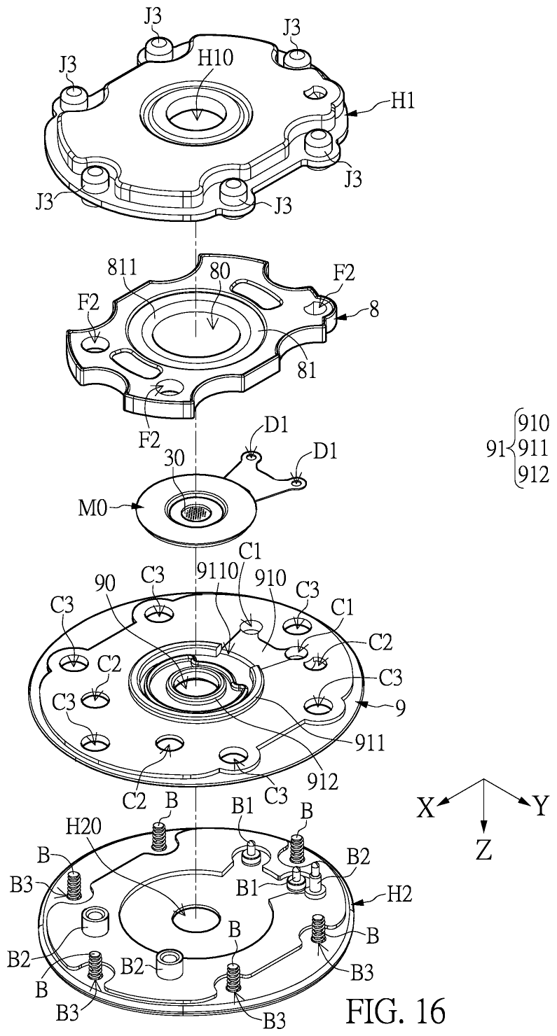

Referring to FIG. 15 and FIG. 16, FIG. 15 is a schematic view of an atomization module according to a sixth embodiment of the present disclosure, and FIG. 16 is a schematic exploded view of the atomization module according to the sixth embodiment of the present disclosure. The present disclosure provides an atomization module M6, which includes the atomization structure M0, a first waterproof member 8, and a second waterproof member 9. The atomization structure M0 includes the base 1, the auxiliary fixing member 2, the atomization member 3, the piezoelectric element 4, and the conductive member 5, and the specific structure thereof is as shown in FIG. 1 and FIG. 2.

The first waterproof member 8 and the piezoelectric element 4 are disposed on opposite sides of the base 1, and the second waterproof member 9 and the piezoelectric element 4 are disposed on the same side of the base 1. The first waterproof member 8 and the second waterproof member 9 can be made of a waterproof soft gel, and the atomization structure M0 can be covered top and bottom by the first waterproof member 8 and the second waterproof member 9 in a separable manner Specifically, the first waterproof member 8 has a first concave portion 81, the first concave portion 81 has a third opening 80, the second waterproof member 9 has a second concave portion 91, and the second concave portion 91 has a fourth opening 90. When the atomization structure M0 is covered top and bottom by the first waterproof member 8 and the second waterproof member 9, the atomization structure M0 is disposed in an accommodating space jointly defined by the first concave portion 81 and the second concave portion 91. The atomization member 3 has a plurality of through holes 30. The through holes 30, the first opening 110, the second opening 20, the third opening 80, and the fourth opening 90 are in spatial communication with each other. Regarding the specific structure of the atomization structure M0, reference can be made to FIG. 1 and FIG. 2.

The first concave portion 81 has a protrusion 811 formed at an inner periphery of the third opening 80, and the protrusion 811 abuts against the base 1. The second concave portion 91 includes an accommodating portion 910, an outer convex wall 911, and an inner convex wall 912. The outer convex wall 911 surrounds the inner convex wall 912, and has a notch 9110. The notch 9110 is opposite to the accommodating portion 910. The inner convex wall 912 corresponds in shape to the piezoelectric element 4. When the atomization structure M0 is disposed in the first concave portion 81, the outer convex wall 911 abuts against the auxiliary fixing member 2, and the inner convex wall 912 abuts against the piezoelectric element 4 but is not in contact with the electrode portions 41. The conductive member 5 is disposed in the accommodating portion 910. The connection portions 51 extend from the notch 9110 to the inside of the outer convex wall 911, and are electrically connected to the electrode portions 41.

The atomization module M6 further includes a first housing H1 and a second housing H2 that are engageable with each other. The first housing H1 and the second housing H2 can be made of a metal member or a plastic member, but are not limited thereto. The atomization structure M0, the first waterproof member 8, and the second waterproof member 9 are disposed in an accommodating space jointly defined by the first housing H1 and the second housing H2. As shown in FIG. 16, the atomization structure M0 is covered top and bottom by the first waterproof member 8 and the second waterproof member 9, and then the atomization structure M0, the first waterproof member 8, and the second waterproof member 9 are together covered top and bottom by the first housing H1 and the second housing H2, so as to achieve a waterproof effect. Furthermore, the first housing H1 has a fifth opening H10, the second housing H2 has a sixth opening H20, and the fifth opening H10 and the sixth opening H20 are in spatial communication with the through holes 30, the first opening 110, the second opening 20, the third opening 80, and the fourth opening 90.

As shown in FIG. 16, the conductive member 5 further has a plurality of limiting holes D1, the accommodating portion 910 of the second waterproof member 9 has a plurality of first limiting holes C1 that correspond to the limiting holes D1, and the second housing H2 includes a plurality of first limiting columns B1 that correspond to the limiting holes D1 and the first limiting holes C1. The first limiting columns B1 respectively penetrate through the limiting holes D1 and the first limiting holes C1, so as to limit the atomization structure M0 and the second waterproof member 9 onto the second housing H2.

The second waterproof member 9 further has a plurality of second limiting holes C2, the first waterproof member 8 further has a plurality of third limiting holes F2, and the second housing H2 further includes a plurality of second limiting columns B2 that respectively correspond to the second limiting holes C2 and the third limiting holes F2. By respectively penetrating through the second limiting holes C2 and the third limiting holes F2, the second limiting columns B2 can limit the first waterproof member 8 and the second waterproof member 9 onto the second housing H2.

Moreover, the second waterproof member 9 has a plurality of fixing holes C3, the first housing H1 has a plurality of first fixing holes J3, and the second housing H2 has a plurality of second fixing holes B3. The fixing holes C3 respectively correspond to the first fixing holes J3 and the second fixing holes B3. When the first housing H1 is engaged with the second housing H2, a plurality of screw columns B respectively penetrate through the fixing holes C3, the second fixing holes B3, and the first fixing holes J3. In this way, the first housing H1 and the second housing H2 can be fixed in position, and the atomization structure M0, the first waterproof member 8, and the second waterproof member 9 between the first housing H1 and the second housing H2 can also be fixed in position.

### Seventh Embodiment

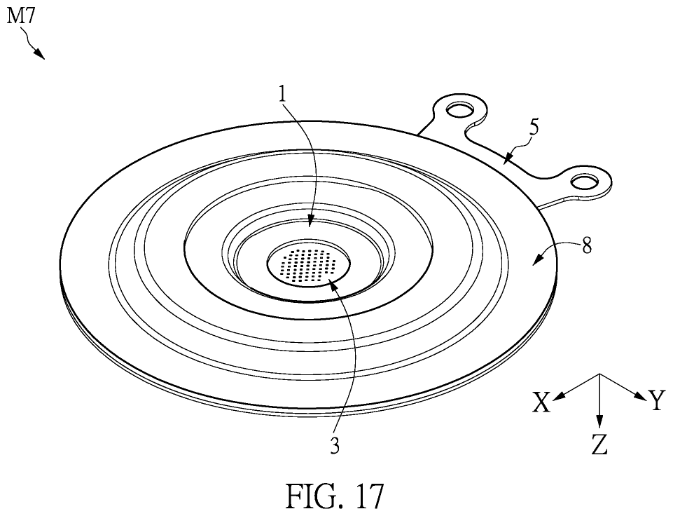

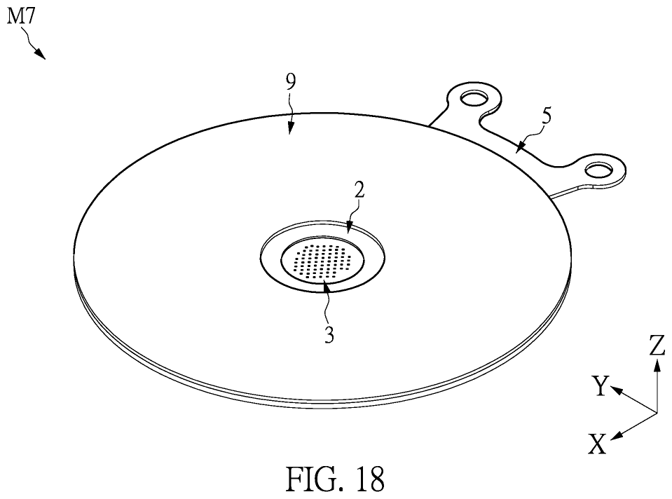

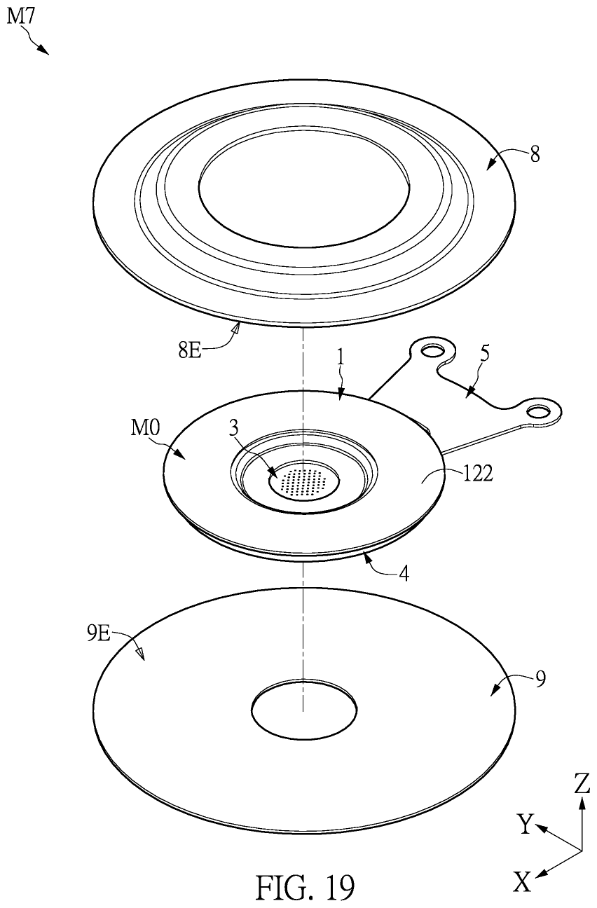

Referring to FIG. 17 to FIG. 19, FIG. 17 is a schematic view of an atomization module according to a seventh embodiment of the present disclosure, FIG. 18 is another schematic view of the atomization module according to the seventh embodiment of the present disclosure, and FIG. 19 is a schematic exploded view of the atomization module according to the seventh embodiment of the present disclosure. The present disclosure provides an atomization module M7, which includes the atomization structure M0, the first waterproof member 8, and the second waterproof member 9. The atomization structure M0 includes the base 1, the auxiliary fixing member 2, the atomization member 3, the piezoelectric element 4, and the conductive member 5, and the specific structure thereof is as shown in FIG. 1 and FIG. 2.

In the seventh embodiment, the first waterproof member 8 and the second waterproof member 9 can be a film that has a gummed surface, such as an adhesive tape. The first waterproof member 8 has a first bonding surface 8E, the second waterproof member 9 has a second bonding surface 9E, and an adhesive (not shown) is disposed on the first bonding surface 8E and the second bonding surface 9E. When the atomization structure M0 is covered top and bottom by the first waterproof member 8 and the second waterproof member 9, the first bonding surface 8E and the second bonding surface 9E both face toward each other and the atomization structure M0. Through the first bonding surface 8E and the second bonding surface 9E, the first waterproof member 8 and the second waterproof member 9 are adhered to each other, and the atomization structure M0 is covered therein. As such, in the seventh embodiment of the present disclosure, the film that has the gummed surface is attached to opposite sides of the atomization structure M0 for sealing the atomization structure M0. In this way, the piezoelectric element 4 inside the atomization structure M0 is blocked from the water vapor of the external environment, so as to achieve the waterproof and insulating effects.

Specifically, the first waterproof member 8 is attached to the second surface 122 of the outer annular portion 12 of the base 1, and is partially attached to the conductive member 5. The second waterproof member 9 is attached to the top surface of the piezoelectric element 4 (i.e., the surface having the electrode portions 41), and is partially attached to the conductive member 5. It is worth mentioning that, the first waterproof member 8 has the third opening 80, the second waterproof member 9 has the fourth opening 90, the atomization member 3 has the through holes 30, and the through holes 30, the first opening 110, the second opening 20, the third opening 80, and the fourth opening 90 are in spatial communication with each other. In other words, the atomization member 3 is not covered by the first waterproof member 8 and the second waterproof member 9, but is exposed from the first waterproof member 8 and the second waterproof member 9.

### BENEFICIAL EFFECTS OF THE EMBODIMENTS

In conclusion, in the atomization modules M1 to M7 provided by the present disclosure, by virtue of “the waterproof member at least partially covering the piezoelectric element” and “the first waterproof member and the second waterproof member being separable from one another, and the atomization structure being covered top and bottom by the first waterproof member and the second waterproof member,” the liquid medicine is prevented from overflowing to the piezoelectric element 4 in the liquid form or the vapor form during vibration of atomization member 3, and short-circuiting of the electrodes on the piezoelectric element 4 does not occur. In addition, through variations of the structure, the atomization modules M1 to M7 of the present disclosure can achieve different effects.

Specifically, by disposing the waterproof member 6 on a single side of the base 1 in the present disclosure, the manufacturing process can be simplified, and the difficulty in the overmolding process of the waterproof member 6 can be reduced. In the present disclosure, the gap between the waterproof member 6 and the atomization structure M0 is configured to be filled with the adhesive, such that the gap is capable of blocking the water vapor, and is prevented from being increased by the thermally-expanded air during the boiling and sanitization process (which may reduce the adhesive strength between the waterproof member 6 and the atomization structure M0, and damage the structure of the atomization module M2). Through the structural design of the groove 70 of the spacer 7 in the present disclosure, the piezoelectric element 4 can have a sufficient space for vibration, and is thus not restricted by the waterproof member 6 when generating vibration. More specifically, in the present disclosure, the waterproof effect can be achieved through configuring the atomization structure M0 to be covered top and bottom by the first waterproof member 8 and the second waterproof member 9.

The foregoing description of the exemplary embodiments of the disclosure has been presented only for the purposes of illustration and description and is not intended to be exhaustive or to limit the disclosure to the precise forms disclosed. Many modifications and variations are possible in light of the above teaching.

The embodiments were chosen and described in order to explain the principles of the disclosure and their practical application so as to enable others skilled in the art to utilize the disclosure and various embodiments and with various modifications as are suited to the particular use contemplated. Alternative embodiments will become apparent to those skilled in the art to which the present disclosure pertains without departing from its spirit and scope.

What is claimed is:

1. An atomization module, comprising:
a base, wherein the base includes a support portion and an outer annular portion, the support portion has a first opening, the outer annular portion surrounds the support portion, and the support portion protrudes relative to the outer annular portion;
an auxiliary fixing member disposed on the support portion, wherein the auxiliary fixing member has a second opening, and the second opening is opposite to the first opening;
an atomization member disposed between the support portion and the auxiliary fixing member;
a piezoelectric element disposed on the outer annular portion, wherein the piezoelectric element and the atomization member are disposed on a same side of the base;
a conductive member electrically connected to the piezoelectric element; and
a waterproof member, wherein the waterproof member at least partially covers the piezoelectric element.

2. The atomization module according to claim 1, wherein the conductive member has two connection portions, two electrode portions are disposed on a surface of the piezoelectric element, and the two connection portions are respectively and electrically connected to the two electrode portions.

3. The atomization module according to claim 2, wherein an inclined support surface is disposed between the support portion and the outer annular portion, the inclined support surface has an inclination angle relative to a horizontal plane, and the inclination angle ranges between 20° C. and 80° C.

4. The atomization module according to claim 3, wherein the waterproof member is disposed on the outer annular portion, and completely covers the piezoelectric element and the two connection portions.

5. The atomization module according to claim 4, wherein the waterproof member is further attached to the inclined support surface.

6. The atomization module according to claim 3, wherein the waterproof member is formed by plating or coating.

7. The atomization module according to claim 3, wherein the waterproof member is disposed on the outer annular portion; wherein the waterproof member completely covers the piezoelectric element and the two connection portions, and partially covers the auxiliary fixing member.

8. The atomization module according to claim 7, further comprising an adhesive filled between the base, the auxiliary fixing member, the piezoelectric element, and the waterproof member.

9. The atomization module according to claim 8, wherein the waterproof member has a third opening, the third opening is in spatial communication with the second opening, and an annular convex wall of the waterproof member is formed along a periphery of the third opening.

10. The atomization module according to claim 2, wherein the waterproof member completely covers the piezoelectric element and the conductive member, and partially covers the base and the auxiliary fixing member.

11. The atomization module according to claim 10, wherein the outer annular portion has a first surface and a second surface that are opposite to each other, and the piezoelectric element and the auxiliary fixing member are disposed on the first surface; wherein, when the waterproof member covers the base, the waterproof member extends from the first surface to the second surface, and partially covers the second surface.

12. The atomization module according to claim 11, wherein the waterproof member is filled between the base, the auxiliary fixing member, and the piezoelectric element; wherein the waterproof member at least covers one half of the second surface, and at least covers one third of a surface of the auxiliary fixing member.

13. The atomization module according to claim 11, further comprising a spacer, wherein the spacer is inserted in the waterproof member, and covers the piezoelectric element.

14. The atomization module according to claim 13, wherein the spacer is filled between the base, the auxiliary fixing member, the piezoelectric element, and the waterproof member, and the two electrode portions are not covered by the spacer.

15. The atomization module according to claim 13, wherein the waterproof member and the spacer are formed by insert molding or overmolding.

16. The atomization module according to claim 2, further comprising a spacer, wherein the spacer is disposed between the waterproof member and the piezoelectric element, and covers the piezoelectric element.

17. The atomization module according to claim 16, wherein the waterproof member includes a first waterproof wall, a second waterproof wall, and a mid-portion, the second waterproof wall surrounds the first waterproof wall, the mid-portion is connected between the first waterproof wall and the second waterproof wall, and a thickness of the mid-portion is less than a thickness of the first waterproof wall and a thickness of the second waterproof wall.

18. The atomization module according to claim 17, wherein the spacer is filled between the first waterproof wall and the second waterproof wall.

19. The atomization module according to claim 16, wherein the two electrode portions are not covered by the spacer.

20. An atomization module, comprising:
an atomization structure, wherein the atomization structure includes:
a base, wherein the base includes a support portion and an outer annular portion, the support portion has a first opening, the outer annular portion surrounds the support portion, and the support portion protrudes relative to the outer annular portion;
an auxiliary fixing member disposed on the support portion, wherein the auxiliary fixing member has a second opening, and the second opening is opposite to the first opening;
an atomization member disposed between the support portion and the auxiliary fixing member;
a piezoelectric element disposed on the outer annular portion, wherein the piezoelectric element and the atomization member are disposed on a same side of the base; and
a conductive member electrically connected to the piezoelectric element; and
a first waterproof member and a second waterproof member, wherein the first waterproof member and the second waterproof member are separable from one another, and the atomization structure is covered top and bottom by the first waterproof member and the second waterproof member.

21. The atomization module according to claim 20, wherein the first waterproof member and the piezoelectric element are disposed on opposite sides of the base, and the second waterproof member and the piezoelectric element are disposed on a same side of the base; wherein the first waterproof member has a first concave portion, the first concave portion has a third opening, the second waterproof member has a second concave portion, and the second concave portion has a fourth opening; wherein, when the atomization structure is covered top and bottom by the first waterproof member and the second waterproof member, the atomization structure is disposed in an accommodating space jointly defined by the first concave portion and the second concave portion.

22. The atomization module according to claim 21, wherein the first concave portion has a protrusion formed at an inner periphery of the third opening, and the protrusion abuts against the base; wherein the second concave portion includes an outer convex wall and an inner convex wall, the outer convex wall abuts against the auxiliary fixing member, and the inner convex wall abuts against the piezoelectric element.

23. The atomization module according to claim 21, further comprising a first housing and a second housing that are engageable with each other, wherein the base, the auxiliary fixing member, the atomization structure, the first waterproof member, and the second waterproof member are disposed in an accommodating space jointly defined by the first housing and the second housing.

24. The atomization module according to claim 23, wherein the atomization member has a plurality of through holes, the first housing has a fifth opening, the second housing has a sixth opening, and the through holes, the first opening, the second opening, the third opening, the fourth opening, the fifth opening, and the sixth opening are in spatial communication with each other.

25. The atomization module according to claim 20, wherein the first waterproof member has a first bonding surface, the second waterproof member has a second bonding surface, and an adhesive is disposed on the first bonding surface and the second bonding surface; wherein, when the atomization structure is covered top and bottom by the first waterproof member and the second waterproof member, the first bonding surface and the second bonding surface both face toward the atomization structure, and the first waterproof member and the second waterproof member are integrally adhered to each other through the adhesive.

26. The atomization module according to claim 25, wherein the first waterproof member has a third opening, the second waterproof member has a fourth opening, the atomization member has a plurality of through holes, and the through holes, the first opening, the second opening, the third opening, and the fourth opening are in spatial communication with each other.

27. The atomization module according to claim 20, wherein the conductive member has two connection portions, two electrode portions are disposed on a surface of the piezoelectric element, and the two connection portions are respectively and electrically connected to the two electrode portions.

28. The atomization module according to claim 20, wherein an inclined support surface is disposed between the support portion and the outer annular portion, the inclined support surface has an inclination angle relative to a horizontal plane, and the inclination angle ranges between 35° C. and 45° C.
